(19) United States
(12) Patent Application Publication
(10) Pub. No.: US 2017/0073733 A1
(43) Pub. Date: Mar. 16, 2017

(54) CARBAPENEMASE AND ANTIBACTERIAL TREATMENT

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); UNIVERSITÉ PARIS - SUD, Orsay (FR)

(72) Inventors: Patrice NORDMANN, Le Kremlin Bicetre (FR); Laurent POIREL, Le Kremlin Bicetre (FR); Laurent DORTET, Le Kremlin Bicetre (FR)

(21) Appl. No.: 15/122,014

(22) PCT Filed: Mar. 6, 2015
(86) PCT No.: PCT/EP2015/054704

§ 371 Date: Aug. 26, 2016

(30) Foreign Application Priority Data
Mar. 7, 2014 (EP) 14305337.9

(51) Int. Cl. C12Q 1/68; C12N 15/115; C07K 16/40; C12N 9/86

(52) U.S. Cl. CPC C12Q 1/689; C12N 9/86; C12N 15/115; C07K 16/40; C12Y 305/02006; C12Q 2600/106; C12Q 2600/158; C12N 2310/16

(57) ABSTRACT

The present invention relates to a carbapenemase and methods using said carbapenemase such as detection methods, screening methods, predictive methods and therapeutic uses.

### FIELD OF THE INVENTION

The present invention relates to carbapenemase and methods using said carbapenemase such as detection methods, screening methods, predictive methods and therapeutic uses.

### BACKGROUND OF THE INVENTION

The discovery and the development of antibacterial compounds has been a real progress in medicine and permitted to save many lives. But the development of antibacterial resistances became a real public health issue. Huge strides forward in health have left the world dangerously complacent. Rising resistance to antibiotics could push overburdened health systems to the brink, while a hyper connected world allows pandemics to spread. This risk case draws on the connections between antibiotic resistance, chronic disease and the failure of the international intellectual property regime, recommending more international collaboration and different funding models”. The risk for Europe was assessed financially at 1.5 billion euros. The severity of this menace is amplified by the fact that research for new antibiotic agents is currently stalled. It may be possible that no totally new agent, active against multiresistant bacteria will be put on the market in a close future.

Today, the understanding of resistance mechanisms and the development of new drugs able to bypass the resistance mechanisms constitute a way of research for the progress in new strategies of treatment for infectious diseases. Enterobacteriaceae are among the most common human pathogens, causing community-acquired as well as hospital-acquired infections. Carbapenem-resistant Enterobacteriaceae have been increasingly reported worldwide since their first identification more than 20 years ago.

Three main classes of carbapenemases have been identified: Ambler class A β-lactamase (KPC), class B (metallo-enzymes), and class D (OXA-48 type).

Klebsiella pneumoniae carbapenemases (KPC) was first reported in the United States in the late 90s and since then worldwide, with a marked endemicity in the United States, Greece, and now Italy. Carbapenemase NDM-1 (New Delhi metallo-β-lactamase-1 (NDM-1) is one of the most recently reported metallo-enzymes. It has spread widely from its reservoir the Indian subcontinent to the other parts of the world. Carbapenemases of the oxacillinase-48 type (OXA-48) have been identified mostly in Mediterranean and southern European countries with a rapid spread.

An early and quick identification of carbapenemase producing infected patients, but also of carriers, is mandatory to prevent the spread of these highly resistant pathogens. The early identification of carriers and implementing of cohorting strategies is the only means to prevent nosocomial outbreaks caused by carbapenemase producers, with very few, if any, therapeutic options.

### SUMMARY OF THE INVENTION

Remarkably, the inventors identified a novel carbapenemase FRI-1 which is the first representant of a new subclass of class A carbapenemase. It shares only 55% amino-acid identity with the most related carbapenemases which are NmcA, IMI-1 and Sme-1/Sme-2.

Accordingly, the invention relates to a carbapenemase comprising or consisting of the amino acid sequence defined by SEQ ID NO: 1 or having at least 80% amino acid sequence identity with the amino acid sequence defined by SEQ ID NO: 1 and a nucleic acid sequence encoding said carbapenemase. The invention also relates to a method for detecting the presence of the FRI-1 carbapenemase of the invention in a biological sample, comprising a step of contacting said biological sample with a binding reagent specific of said carbapenemase.

The invention also relates to an antibody or an aptamer which specifically binds to the FRI-1 carbapenemase according to the invention.

The invention also relates to a method for detecting the presence of the nucleic acid sequence encoding a carbapenemase of the invention in a biological sample, comprising a step of contacting said biological sample with a nucleic acid molecule which specifically hybridizes to said nucleic acid sequence.

The invention also relates to a probe or a set of primers which specifically hybridizes to the nucleic acid sequence encoding the FRI-1 carbapenemase according to the invention.

The invention also relates to a method for screening an antibacterial substance comprising the step of determining the ability of a candidate substance to inhibit the activity of a purified carbapenemase of the invention.

The invention further relates to a method for determining whether a microorganism is resistant or possesses a decreased susceptibility to a β-lactam compound (in particularly to carbapenems) comprising the step of detecting in said microorganism the presence of a nucleic acid encoding a carbapenemase or the corresponding protein of the invention wherein the presence of said nucleic acid is indicative that said microorganism is resistant to β-lactams including carbapenems.

### DETAILED DESCRIPTION OF THE INVENTION

### Definitions

By “purified” and “isolated” it is meant, when referring to a polypeptide or a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

As used herein, the term “subject” refers to a human or another mammal (e.g., primate, dog, cat, goat, horse, pig, mouse, rat, rabbit, and the like), that can be infected with a strain. In a particular embodiment of the present invention, the subject is a human. In particular, the subject can be a patient being infected with a bacteria that is resistant or has intermediate susceptibility to β-lactams (including carbapenems) or who can be just a carrier of such a strain.

In its broadest meaning, the term “treating” or “treatment” refers to reversing, alleviating, inhibiting the progress of the disorder or condition to which such term applies, or one or more symptoms of such disorder or condition.

“Pharmaceutically” or “pharmaceutically acceptable” refer to molecular entities and compositions that do not produce an adverse, allergic or other untoward reaction when administered to a mammal, especially human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The term “β-lactam” has its general meaning in the art and refers to a broad class of antibiotics that include penicillin derivatives, cephalosporins, monobactams, carbapenems, and β-lactam molecules action as β-lactamase inhibitors. Said family of antibiotics is characterised by a β-lactam nucleus (see the formula below) in its molecular structure:

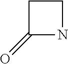

β-lactam compounds include, but are not limited to, imipenem, meropenem, ertapenem, faropenem, doripenem and panipenem.

The term “carbapenemase” has its general meaning in the art and refers to a class of enzymes produced by some bacteria belonging to the β-lactamase family. Said enzymes may be responsible for resistance to β-lactam antibiotics like oxyiminocephalosporins, cephamycins and carbapenems by hydrolyzing β-lactam cycle of said antibiotics.

As used herein, the term “biological sample” may refer to a sample derived from a subject, for example bodily fluids such as blood or urine, or throat swabs, nasal swabs, dermal swabs, sputum, feces or bronchial aspirates. The biological sample may also mean cultures of bacteria from various environments. Typically the bacterial cultures may be prepared from biological samples by plating and growing the bacteria.

### Enzymes and Nucleic Acids of the Invention

The inventors have identified and isolated a carbapenemase herein after named FRI-1 which hydrolyses all β-lactams. FRI-1 has ca. 55% amino acid identity with NmcA, IMI and SME. It hydrolyses preferentially benzylpenicillin, ticarcillin and carbapenems. Its activity is weakly inhibited by known class A inhibitors such as clavulanic acid.

Thus, a first object of the invention relates to a carbapenemase comprising or consisting of the amino acid sequence defined by SEQ ID NO: 1 (as follows):

TABLE 1
Amino acid sequence of the carbapenemase
FRI-1 (SEQ ID NO: 1).

MFFFKKGASTFIFLLCLPLNSFASQVINSVEEMRELETSFGGRIGVYILN
PKNGKEFAYRQDERFPLCSSFKAFLAASVLKRTQEKSVSLDDMVEYSGRV
MEKHSPVSEKYRKTGASVQTLAKAAIQYSDNGASNLLMERYIGGPEGLTA
FMRSTGDTDFRLDRWELELNTAIPGDERDTSTPKAVAMSLKNIAFGSVLD
AKNKSLLQEWLKGNTTGNARIRAAVPDKWVVGDKTGTCGFYGTANDVAIL
WTDANSPAVMAVYTTRPNQNDKHDEAVIKNAAKIAIKAVYGSY

In one embodiment, the carbapenemase has a sequence at least 80%, 85%, 90%, 95%, 96%, 97%, 98% or 99% identical to the sequence SEQ ID NO: 1.

A further object of the invention relates to a nucleic acid sequence encoding a carbapenemase of the invention. In a particular embodiment, the invention relates to a nucleic acid sequence encoding the FRI-1 carbapenemase defined by SEQ ID NO: 2 (as follows):

TABLE 2
Nucleic acid sequence of bla FRI-1 (SEQ ID NO: 2).

ATGTTTTTTTTTAAAAAAGGTGCAAGTACATTTATTTTTTTGCTCTGTCT
TCCATTGAACTCATTCGCCTCTCAGGTAATTAATAGTGTTGAGGAAATGA
GGGAATTGGAAACTTCTTTTGGGGGGCGGATAGGTGTTTATATTTTAAAC
CCAAAAAATGGGAAAGAATTTGCCTACAGACAAGATGAGAGATTTCCTTT
ATGTAGTTCATTTAAGGCGTTCCTCGCTGCATCCGTATTAAAAAGAACTC
AGGAGAAATCTGTTTCTCTTGATGATATGGTGGAATATTCTGGACGTGTT
ATGGAAAAGCATTCTCCTGTGTCAGAAAAATACCGTAAAACAGGAGCAAG
CGTGCAGACTTTGGCCAAGGCAGCAATTCAGTATAGTGACAATGGAGCTT
CTAATCTATTAATGGAAAGATACATAGGAGGTCCTGAGGGTTTGACTGCA
TTTATGCGGTCAACGGGAGACACTGACTTCAGGCTTGATCGTTGGGAATT
AGAATTAAACACAGCTATTCCAGGCGATGAACGAGATACTTCAACTCCAA
AAGCAGTGGCAATGAGCCTTAAAAATATTGCTTTTGGTTCAGTACTCGAT
GCTAAAAATAAATCATTGCTGCAGGAATGGCTTAAAGGCAACACTACTGG
TAATGCGCGAATTAGAGCTGCGGTTCCAGATAAGTGGGTTGTTGGCGATA
AAACAGGCACCTGTGGTTTTTATGGTACAGCCAATGATGTTGCTATTTTA
TGGACAGACGCCAATTCACCTGCAGTTATGGCTGTCTACACAACACGTCC
TAATCAAAACGACAAACATGACGAAGCAGTAATTAAAAATGCTGCAAAAA
TAGCTATAAAGGCAGTTTATGGAAGTTATAA

A carbapenemase of the invention can be produced as a recombinant protein. For obtaining a recombinant form of a carbapenemase of the invention, or a biologically active fragment thereof, the one skilled in the art may insert the nucleic acid encoding the corresponding polypeptide (SEQ ID NO: 1), e.g. into a suitable expression vector and then transform appropriate cells with the resulting recombinant vector.

Methods of genetic engineering for producing the polypeptides having a carbapenemase activity according to the invention under the form of recombinant polypeptides are well known from the one skilled in the art.

As it is well known from the one skilled in the art, the recombinant vector preferably contains a nucleic acid that enables the vector to replicate in one or more selected host cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression and cloning vectors usually contain a promoter operably linked to the nucleic acid sequence encoding the polypeptide of interest to direct mRNA synthesis. Promoters recognized by a variety of potential host cells are well known. Promoters suitable for use with prokaryotic hosts include the β-lactamase and lactose promoter systems (Chang et al., 1978; Goeddel et al., 1979), alkaline phosphatase, a tryptophan (trp) promoter system (Goeddel, 1980; EP 36,776), and hybrid promoters such as the tac promoter (deBoer et al., 1983). Promoters for use in bacterial systems also will contain a Shine-Dalgarno (S.D.) sequence operably linked to the DNA encoding the polypeptide of interest.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding polypeptide of interest.

Illustratively, a recombinant vector having inserted therein a nucleic acid encoding a polypeptide of interest according to the invention having a carbapenemase activity may be transfected to bacterial cells in view of the recombinant polypeptide production, e.g. E. coli cells as shown in the examples herein.

Then, the recombinant polypeptide of interest having a carbapenemase activity may be purified, e.g. by one or more chromatography steps, including chromatography steps selected from the group consisting of affinity chromatography, ion exchange chromatography and size exclusion chromatography.

Illustratively, the recombinant polypeptide of interest having a carbapenemase activity may be purified by performing a purification method comprises (a) a step of affinity chromatography, e.g. on a Ni2+-nitriloacetate-agarose resin, (b) a step of anion exchange chromatography with the eluate of step (a) and (c) a size exclusion chromatography with the eluate of step (b).

The purified recombinant polypeptide of interest having a carbapenemase activity may be subjected to a concentration step, e.g. by ultrafiltration, before being stored in an appropriate liquid solution, e;g. at a temperature of −20° C.

Alternatively, a recombinant polypeptide of interest having a carbapenemase activity may be produced by known methods of peptide synthesis. For instance, the polypeptide sequence of interest, or portions thereof, may be produced by direct peptide synthesis using solid-phase techniques. (See, e.g., Stewart et al., 1969; Merrifield, 1963). In vitro protein synthesis may be performed using manual techniques or by automation. Automated synthesis may be accomplished, for instance, with an Applied Biosystems Peptide Synthesizer (Foster City, Calif.) using manufacturer's instructions. Various portions of the polypeptide of interest may be chemically synthesized separately and combined using chemical or enzymatic methods to produce the full-length polypeptide of interest.

### Methods of Screening of the Invention

A further object of the invention relates to a method for screening an antibacterial substance comprising the step of determining the ability of a candidate substance to inhibit the activity of a purified carbapenemase of the invention.

In a particular embodiment, said method comprises the steps of:
(i) providing a carbapenemase of the invention and a substrate thereof;
(ii) bringing the candidate substance to be tested into contact with the carbapenemase and the substrate of step i);
(iii) determining the activity of said carbapenemase in presence of the candidate substance to be tested;
(iv) comparing the activity of said carbapenemase determined at step iii) with the activity of said carbapenemase in the absence of said candidate substance;
(v) and positively selecting the candidate substance that inhibits the catalytic activity of said carbapenemase.

As intended herein, a candidate substance to be tested inhibits the catalytic activity of said carbapenemase if the activity of the said enzyme determined in presence of said candidate substance is lower than the activity of the said enzyme determined in absence of said candidate substance.

In a particular embodiment of the screening method described above, the catalytic activity of the carbapenemase of the invention is assessed using as a substrate a molecule of the class of β-lactams except aztreonam. Preferably, said molecule is selected from the group of ticarcillin, piperacillin-tazobactam, imipenem, meropenem, ceftazidime and cefepime and more preferably from the group of ticarcillin, piperacillin-tazobactam, imipenem and meropenem.

Accordingly, the catalytic activity of said carbapenemase is determined by detecting or quantifying the formation of a derivative of β-lactam molecule that results from the opening β-lactam ring as determined by detection of this opened derivative by UV spectrophotometry.

Preferably, the candidate substances that are positively selected at step (v) of the method above are those that cause a decrease of the hydrolyze of the beta-lactam cycle of β-lactams that leads to less than 0.5 times the hydrolyze rate of the same enzyme in the absence of the candidate substance, more preferably a decrease that leads to less 0.3, 0.2, 0.1, 0.05 or 0.025 times the hydrolyze rate of the same enzyme in the absence of the candidate substance. The most active candidate substances that may be positively selected at step (v) of the method above may completely block the catalytic activity of said enzyme, which leads to an hydrolyze rate of beta-lactam cycle which is undetectable, i.e. zero, or very close to zero.

As detailed previously in the specification, this invention encompasses methods for the screening of candidate antibacterial substances that inhibit the activity of a carbapenemase as defined herein.

However, this invention also encompasses methods for the screening of candidate antibacterial substances that are based on the ability of said candidate substances to bind to a carbapenemase as defined herein, thus methods for the screening of potentially antibacterial substances.

The binding assays can be performed in a variety of formats, including protein-protein binding assays, biochemical screening assays, immunoassays, and cell-based assays, which are well characterized in the art.

All binding assays for the screening of candidate antibacterial substances are common in that they comprise a step of contacting the candidate substance with a carbapenemase as defined herein, under conditions and for a time sufficient to allow these two components to interact.

These screening methods also comprise a step of detecting the formation of complexes between said carbapenemase and said candidate antibacterial substances.

Thus, screening for antibacterial substances includes the use of two partners, through measuring the binding between two partners, respectively a carbapenemase as defined herein and the candidate compound.

In binding assays, the interaction is binding and the complex formed between a carbapenemase as defined above and the candidate substance that is tested can be isolated or detected in the reaction mixture. In a particular embodiment, the carbapenemase as defined above or alternatively the antibacterial candidate substance is immobilized on a solid phase, e.g., on a microtiter plate, by covalent or non-covalent attachments. Non-covalent attachment generally is accomplished by coating the solid surface with a solution of the carbapenemase of the invention and drying. Alternatively, an immobilized antibody, e.g., a monoclonal antibody, specific for the carbapenemase of the invention to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

The binding of the antibacterial candidate substance to a carbapenemase of the invention may be performed through various assays, including traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, 1989; Chien et al., 1991) as disclosed by Chevray and Nathans, 1991. Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for .beta.-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Thus, another object of the invention consists of a method for the screening of antibacterial substances, wherein said method comprises the steps of:
(i) providing a candidate substance;
(ii) assaying said candidate substance for its ability to bind to a carbapenemase of the invention;

The same method may also be defined as a method for the screening of antibacterial substances, wherein said method comprises the steps of:
(i) contacting a candidate substance with a carbapenemase of the invention;
(ii) detecting the complexes eventually formed between said carbapenemase and said candidate substance.

The candidate substances, which may be screened according to the screening method above, may be of any kind, including, without being limited to, natural or synthetic compounds or molecules of biological origin such as polypeptides.

Inhibitor substances positively selected at the end of the in vitro screening methods as described above are inhibitors of a carbapenemase of the invention. Accordingly, the activity of selected candidate can be studied by assaying the antibacterial activity of a combination of such compounds with a β-lactam compound against gram negative bacteria expressing a carbapenemase of the invention.

Particularly, the β-lactam compounds which can be used in combination with said inhibitor substances are β-lactams which are hydrolyzed by the carbapenemases of the invention such as ticarcillin, piperacillin-tazobactam, imipenem, meropenem, ertapenem, ceftazidime and cefepime.

An example of bacterial strain expressing a carbapenemase of the invention is Pseudomonas stutzeri. Thus, the antibacterial activity of a combination of an inhibitor substance with a β-lactam compound can be tested against this Gram-negative bacterial strain.

Inhibitor substances that have been positively selected at the end of any one of the in vitro screening methods of the invention may then be assayed for their ex vivo activity in combination with a β-lactam compound, in a further stage of their selection as a useful antibacterial active ingredient of a pharmaceutical composition.

By “ex vivo” antibacterial activity, it is intended herein the antibacterial activity of the combination of a positively selected candidate compound and a β-lactam compound against bacterial cells expressing a carbapenemase of the invention that are cultured in vitro.

Thus, any substance that has been shown to behave like an inhibitor of a carbapenemase, after positive selection at the end of any one of the in vitro screening methods that are disclosed previously in the present specification, may be further assayed for his ex vivo antibacterial activity against bacterial cells expressing a carbapenemase of the invention.

Consequently, any one of the screening methods that are described above may comprise a further step of assaying a combination with a positively selected inhibitor substance and a β-lactam compound for its ex vivo antibacterial activity.

Usually, said further step consists of preparing in vitro cultures of bacterial cells expressing a carbapenemase of the invention and then adding to said bacterial cultures the combination to be tested, before determining the ability of said candidate compound to block bacterial growth or even most preferably kill the cultured bacterial cells.

Typically, bacterial cells are plated in Petri dishes containing the appropriate culture medium, generally in agar gel, at a cell number ranging from 10 to 10³ bacterial cells, including from 10 to 10² bacterial cells. In certain embodiments, serials of bacterial cultures are prepared with increasing numbers of seeded bacterial cells.

Typically, the combination to be tested is then added to the bacterial cultures, preferably with a serial of amounts of said candidate compounds for each series of a given plated cell number of bacterial cultures.

Then, the bacterial cultures are incubated in the appropriate culture conditions, most preferably starvation conditions, for instance in a cell incubator at the appropriate temperature, and for an appropriate time period, for instance a culture time period ranging from 1 day to 4 days, before counting the resulting CFUs (Colony Forming Units), either manually under a light microscope or binocular lenses, or atomically using an appropriate apparatus.

Generally, appropriate control cultures are simultaneously performed i.e; negative control cultures without the combination and positive control cultures with an antibiotic that is known to be toxic against the cultured bacterial cells (such as aztreonam or any β-lactam molecule that are not hydrolyzed by a carbapenemase of the invention).

Finally, said candidate compound is positively selected at the end of the method if it reduces the number of CFUs, as compared with the number of CFUs found in the corresponding negative control cultures.

Thus, another object of the present invention consists of a method for the ex vivo screening of a candidate antibacterial substance which comprises the steps of:

a) selecting a candidate substance by performing the in vitro screening of the invention; and

b) assaying said candidate substance positively selected at the end of step a) for its ex vivo antibacterial activity.

Inhibitor substances that have been positively selected at the end of any one of the screening methods that are previously described in the present specification may then be assayed for their in vivo antibacterial activity in combination with a β-lactam compound, in a further stage of their selection as a useful antibacterial active ingredient of a pharmaceutical composition.

As explained above, the compound is tested in combination with a β-lactam compound against bacterial cells expressing a carbapenemase of the invention.

Thus, any substance that has been shown to behave like an inhibitor of a carbapenemase, after positive selection at the end of any one of the screening methods that are disclosed previously in the present specification, may be further assayed for his in vivo antibacterial activity.

Consequently, any one of the screening methods that are described above may comprise a further step of assaying the combination of a positively selected inhibitor substance and a β-lactam substance for its in vivo antibacterial activity.

Usually, said further step consists of administering said combination to a mammal and then determining the antibacterial activity of said combination.

Mammals are preferably non-human mammals, at least at the early stages of the assessment of the in vivo antibacterial effect of the combination tested. However, at further stages, human volunteers may be administered with said combination to confirm safety and pharmaceutical activity data previously obtained from non-human mammals.

Non-human mammals encompass rodents like mice, rats, rabbits, hamsters, guinnea pigs. Non-human mammals and also cats, dogs, pigs, calves, cows, sheeps, goats. Non-human mammals also encompass primates like macaques and baboons.

Thus, another object of the present invention consists of a method for the in vivo screening of a candidate antibacterial substance which comprises the steps of:

a) performing a method for the in vitro screening of a antibacterial substances as disclosed in the present specification, with a candidate substance; and

b) assaying a candidate substance that has been positively selected at the end of step a) in combination with a β-lactam substance for its in vivo antibacterial activity.

Preferably, serial of doses containing increasing amounts of the inhibitor substance are prepared in view of determining the antibacterial effective dose of said inhibitor substance (when used in combination with a β-lactam compound) in a mammal subjected to a bacterial infection, typically a Gram (−) bacterial infection. Generally, the ED₅₀ dose is determined, which is the amount of the inhibitor substance that makes the combination effective against a bacterial strain expressing a carbapenemase of the invention in 50% of the animals tested. In some embodiments, the ED₅₀ value is determined for various distinct bacteria species, in order to assess the spectrum of the antibacterial activity.

In certain embodiments, it is made use of serial of doses of the inhibitor substance tested ranging from 1 ng to 10 mg per kilogram of body weight of the mammal that is administered therewith.

Several doses may comprise high amounts of said inhibitor substance, so as to assay for eventual toxic or lethal effects of said inhibitor substance and then determine the LD₅₀ value, which is the amount of said inhibitor substance that is lethal for 50% of the mammal that has been administered therewith.

β-Lactam compound is used at the normal dose actually used in antibacterial treatment. Illustratively, the daily amount of imipenem to be administered to an adult patient weighing 80 kg will typically ranges from 1 g to 4 g. Illustratively, the daily amount of meropenem, ertapenem, faropenem, doripenem or panipenem to be administered to an adult patient weighing 80 kg will typically be of about 1-2 g.

According to the invention, the inhibitor substance in combination with a β-lactam compound forms an antibacterial composition.

The antibacterial composition to be assayed may be used alone under the form of a solid or a liquid composition.

When the antibacterial composition is used alone, the solid composition is usually a particulate composition of said antibacterial composition, under the form of a powder.

When the antibacterial composition is used alone, the liquid composition is usually a physiologically compatible saline buffer, like Ringer's solution or Hank's solution, in which said antibacterial composition is dissolved or suspended.

In other embodiments, said antibacterial composition is combined with one or more pharmaceutically acceptable excipients for preparing a pre-pharmaceutical composition that is further administered to a mammal for carrying out the in vivo assay.

Before in vivo administration to a mammal, the antibacterial composition selected through any one of the in vitro screening methods above may be formulated under the form of pre-pharmaceutical compositions. The pre-pharmaceutical compositions can include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the test composition or formulation may also include other carriers, adjuvants, or non-toxic, non-therapeutic, non-immunogenic stabilizers and the like.

Compositions comprising such carriers can be formulated by well known conventional methods. These test compositions can be administered to the mammal at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by taking into account, notably, clinical factors. As is well known in the medical arts, dosages for any one mammal depends upon many factors, including the mammal's size, body surface area, age, the particular compound to be administered, sex, time and route of administration and general health. Administration of the suitable pre-pharmaceutical compositions may be effected by different ways, e.g., by intravenous, intraperitoneal, subcutaneous, intramuscular, topical or intradermal administration. If the regimen is a continuous infusion, it should also be in the range of 1 ng to 10 mg units per kilogram of body weight per minute, respectively. Progress can be monitored by periodic assessment. The pre-pharmaceutical compositions of the invention may be administered locally or systemically. Administration will generally be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, anti-oxidants, chelating agents, and inert gases and the like.

The antibacterial composition may be employed in powder or crystalline form, in liquid solution, or in suspension.

The injectable pre-pharmaceutical compositions may take such forms as suspensions, solutions, or emulsions in oily or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically comprised of sterile water, saline, or another injectable liquid, e.g., peanut oil for intramuscular injections. Also, various buffering agents, preservatives and the like can be included.

Topical applications may be formulated in carriers such as hydrophobic or hydrophilic base formulations to provide ointments, creams, lotions, in aqueous, oleaginous, or alcoholic liquids to form paints or in dry diluents to form powders.

Oral pre-pharmaceutical compositions may take such forms as tablets, capsules, oral suspensions and oral solutions. The oral compositions may utilize carriers such as conventional formulating agents and may include sustained release properties as well as rapid delivery forms.

In certain embodiments of the in vivo screening assay, the antibacterial composition is administered to a mammal which is the subject of a bacterial infection. For non-human mammals, these animals have been injected with a composition containing bacteria prior to any administration of the inhibitor compound.

In certain other embodiments of the in vivo screening assay, non-human animals are administered with the inhibitor compound to be tested prior to being injected with a composition containing bacteria.

Generally, non-human mammals are injected with a number of bacterial cells expressing a carbapenemase of the invention cells ranging from 1×10² to 1×10¹² cells, including from 1×10⁶ to 1×10⁹ cells. In some embodiments, bacterial cells expressing a carbapenemase of the invention cells in an in vitro-generated dormant state are used for injection.

Generally, bacteria cells that are injected to the non-human mammals are contained in a physiologically acceptable liquid solution, usually a saline solution like Ringer's solution or Hank's solution.

Generally, in the embodiment wherein the inhibitor compound to be tested is administered subsequently to bacterial inoculation, said inhibitor compound is administered form 1 hour to 96 hours after bacterial injection, including from 6 hours to 48 hours after bacterial injection.

Generally, in the embodiment wherein the inhibitor compound to be tested is administered prior to bacterial injection, said inhibitor compound is administered from 1 min to 3 hours prior to bacterial injection.

Generally, all animals are sacrificed at the end of the in vivo assay.

For determining the in vivo antibacterial activity of the inhibitor compound that is tested, blood or tissue samples of the tested animals are collected at determined time periods after administration of said inhibitor compound and bacteria counts are performed, using standard techniques, such as staining fixed slices of the collected tissue samples or plating the collected blood samples and counting the bacterial colonies formed.

Then, the values of the bacteria counts found for animals having been administered with increasing amounts of the inhibitor compound tested are compared with the value(s) of bacteria count(s) obtained from animals that have been injected with the same number of bacteria cells but which have not been administered with said inhibitor compound.

As already disclosed earlier in the present specification, various β-lactam candidate compounds have been assayed with the screening method of the invention and have been positively selected as compounds having a great potential value for treating individuals who have been infected by a bacterial strain expressing a carbapenemase of the invention.

Another object of the invention relates to an inhibitor of a carbapenemase of the invention in association with a β-lactam compound for an antibacterial treatment.

The invention also relates to an antibacterial composition containing an inhibitor of a carbapenemase of the invention and a β-lactam compound for an antibacterial treatment.

This invention also pertains to a method for treating individuals infected by Gram-negative bacteria expressing a carbapenemase of the invention comprising a step of administering to the said individuals an effective amount of an antibacterial composition of the invention.

Preferably, said antibacterial comprises one or more pharmaceutically acceptable excipient(s).

Such antibacterial compositions are under the form of dosage forms adapted for a daily administration of an amount of β-lactam of at least 1 mg and up to 10 g.

The effective amount of each component of antibacterial composition may be easily adapted by the one skilled in the art, depending notably on the age and of the weight individual to be treated.

The daily amount of each component of antibacterial composition may be administered to the patient through one or more uptakes, e.g. from one to six uptakes.

### Kits of the Invention

The present invention also relates to compositions or kits for the screening of antibacterial substances.

In certain embodiments, said compositions or kits comprise a purified carbapenemase of the invention, preferably under the form of a recombinant protein.

In said compositions or said kits, said carbapenemase may be under a solid form or in a liquid form. Solid forms encompass powder of said carbapenemase under a lyophilized form. Liquid forms encompass standard liquid solutions known in the art to be suitable for protein long time storage.

Preferably, said carbapenemase is contained in a container such as a bottle, e.g. a plastic or a glass container. In certain embodiments, each container comprises an amount of said carbapenemase ranging from 1 ng to 10 mg, either in a solid or in a liquid form.

Further, said kits may comprise also one or more reagents, typically one or more substrate(s), necessary for assessing the enzyme activity of said carbapenemase.

Illustratively, if said kit comprises a container of carbapenemase, then said kit may also comprise a container comprising an appropriate amount of the substrate.

In certain embodiments, a kit according to the invention comprises one or more of each of the containers described above.

In another embodiment, said kits or compositions of the invention may also comprise a β-lactam compound for assessing the activity of the inhibitors selected by the screening methods of the invention. Particularly, said β-lactam compound can be selected among the group of benzylpenicillin, ticarcillin, piperacillin-tazobactam, imipenem, meropenem, ceftazidime and cefepime.

### Detection Methods of the Invention

The invention also relates to a method for detecting the presence of a nucleic acid encoding the FRI-1 carbapenemase of the invention or a protein encoded by said nucleic acid.

Thus, another object of the invention relates to a method for detecting the presence of the FRI-1 carbapenemase of the invention in a biological sample, comprising a step of contacting said biological sample with a binding reagent specific of said carbapenemase (SEQ ID NO: 1).

The term “binding reagent” according to the invention may be any binding compound that is able to specifically bind to the carbapenemase of the invention.

In a particular embodiment, a biological sample, such as a body fluid obtained from a subject, may be contacted with antibodies specific of FRI-1, i.e. antibodies that are capable of distinguishing between FRI-1 and any other related protein such as NmcA, KPC, Sme and IMI.

Antibodies that specifically recognize FRI-1 also make part of the invention. The antibodies are specific FRI-1 i.e. they do not cross-react with any other carbapenemase.

The antibodies of the present invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering FRI-1 subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988) which is hereby incorporated in the references.

A “monoclonal antibody” in its various grammatical forms refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified FRI-1 into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Antibodies raised against FRI-1 may be cross reactive with other carbapenemases. Accordingly a selection of antibodies specific for FRI-1 is required. This may be achieved by depleting the pool of antibodies from those that are reactive with the FRI-1, for instance by submitting the raised antibodies to an affinity chromatography against FRI-1.

Alternatively, binding reagents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

It should be intented that the present invention encompasses a kit comprising antibody or an aptamer for detecting the carbapenemase of the invention in particular an antibody or an aptamer specific for the FRI-1 carbapenemase.

The invention also relates to a method for detecting the gene of the FRI-1 carbapenemase of the invention in a biological sample, comprising a step of contacting said biological sample with a nucleic acid sequence which specifically hybridizes with (such as probes or primers) the nucleic acid sequence encoding the FRI-1 carbapenemase of the invention. The entire bla FRI-1 may be used for detecting this gene.

In the context of the invention, bla FRI-1 nucleic acid molecules include mRNA, genomic DNA and cDNA derived from mRNA. DNA or RNA can be single stranded or double stranded. These may be utilized for detection by amplification and/or hybridization with a probe, for instance.

The nucleic acid sample may be obtained from any biological sample, such as a body fluid. Body fluids include blood, plasma, serum, lymph, urine etc. DNA may be extracted using any methods known in the art, such as described in Sambrook et al., 1989. RNA may also be isolated, for instance from tissue biopsy, using standard methods well known to the one skilled in the art such as guanidium thiocyanate-phenol-chloroform extraction.

The bla FRI-1 gene may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of the bla FRI-1 gene. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify the bla FRI-1 sequence.

Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

Therefore, useful nucleic acid molecules, in particular oligonucleotide probes or primers, according to the present invention include those which specifically hybridize to the gene encoding the carbapenemase of the invention.

Oligonucleotide probes or primers may contain at least 10, 15, 20 or 30 nucleotides. Their length may be shorter than 400, 300, 200 or 100 nucleotides.

It should be further noted that probes, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probes, primers, aptamers or antibodies of the invention, is intended to encompass direct labelling of the probes, primers, aptamers or antibodies of the invention by coupling (i.e., physically linking) a detectable substance to the probes, primers, aptamers or antibodies of the invention, as well as indirect labelling of the probes, primers, aptamers or antibodies of the invention by reactivity with another reagent that is directly labeled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labeled secondary antibody and end-labeling of a DNA probe with biotin such that it can be detected with fluorescently labeled streptavidin. An antibody or aptamer of the invention may be labelled with a radioactive molecule by any method known in the art. For example radioactive molecules include but are not limited radioactive atom for scintigraphic studies such as I123, I124, In111, Re186, Re188.

It should be intented that the present invention encompasses a kit comprising a set of primer pairs for identifying the carbapenemase gene of the invention causing carbapenem resistance in bacteria and in particular a set of primer pairs for detecting the presence of the FRI-1 carbapenemase of the invention as previously mentioned.

### Predictive Methods of the Invention

The inventors have shown that the carbapenemase FRI-1 is responsible for a resistance mechanism against compounds of the family of β-lactams.

Thus, a further object of the invention relates to a method for determining whether a microorganism is resistant to a β-lactam compound comprising the step of detecting in said microorganism the presence of a nucleic acid encoding a carbapenemase of the invention wherein the presence of said nucleic acid is indicative that said microorganism is resistant to β-lactams.

In a particular embodiment, the step of detecting the presence of a nucleic acid encoding a carbapenemase of the invention is carried out as previously described.

The present invention relates to a method for determining whether a microorganism has an extended activity (e.g. an increased hydrolytic activity towards carbapenems) to a β-lactam compound comprising the step of detecting whether any nucleotide may increase the rate of hydrolysis to carbapenems or cephalosporins by this microorganism.

The presence of said nucleic acid can be assayed by detecting the DNA sequence of a carbapenemase of the invention in the genome of the microorganism of interest or by detecting the expression of said nucleic acid, at mRNA or protein level in a sample containing said microorganism.

Methods of detecting the presence of a nucleic acid in a microorganism are well known in the art. Typically, said the nucleic acids of the microorganism may be subjected to amplification by polymerase chain reaction (PCR), using specific oligonucleotide primers that enable amplification of a region in the nucleic acid encoding for the carbapenemase of the invention. Said amplified sequences may be detected by hybridization with a suitable probe or by direct sequencing, or any other appropriate method known in the art.

In a particular embodiment of the invention, the presence of a gene encoding for a carbapenemase of the invention can be assayed using the pair of specific primers defined by the sense and the nucleic acid sequence and the antisense primers.

The expression of the nucleic acid encoding for a carbapenemase of the invention can be assayed by detecting the mRNA or protein encoded by said nucleic acid.

Methods for detecting mRNA are well known in the art. For example, the nucleic acid contained in the samples containing the microorganism of interest is first extracted according to standard methods, for example using lytic enzymes or chemical solutions or extracted by nucleic-acid-binding resins following the manufacturer's instructions. The extracted mRNA may be then detected by hybridization (e. g., Northern blot analysis).

Alternatively, the extracted mRNA may be subjected to coupled reverse transcription and amplification, such as reverse transcription and amplification by polymerase chain reaction (RT-PCR), using specific oligonucleotide primers that enable amplification of a region in the nucleic acid of a carbapenemase of the invention may be used. Quantitative or semi-quantitative RT-PCR is preferred. Real-time quantitative or semi-quantitative RT-PCR is particularly advantageous. Extracted mRNA may be reverse transcribed and amplified, after which amplified sequences may be detected by hybridization with a suitable probe or by direct sequencing, or any other appropriate method known in the art.

Other methods of Amplification include ligase chain reaction (LCR), transcription mediated amplification (TMA), strand displacement amplification (SDA) and nucleic acid sequence based amplification (NASBA).

Determination of the expression of a nucleic acid of interest can be easily assayed by detection of the protein encoded by said nucleic acid.

Thus, the invention also relates to a method for determining whether a microorganism is resistant or of intermediate susceptibility to a β-lactam compound (including carbapenems) comprising the step of detecting in said microorganism the presence of the carbapenemase encoded by said nucleic acid wherein the presence of said protein is indicative that said microorganism is resistant or of intermediate susceptibility to a β-lactam compound.

In a particular embodiment, the step of detecting the presence of the carbapenemase of the invention is carried out as previously described.

Such methods comprise contacting a sample susceptible of containing said nucleic acid (so, according to the invention, containing the microorganism of interest) with a binding partner capable of selectively interacting with the protein of interest present in the sample. The binding partner is generally an antibody that may be polyclonal or monoclonal, preferably monoclonal.

The presence of the said protein can be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, immunocytochemistry, immunohistochemistry, etc. The reactions generally include revealing labels such as fluorescent, chemiluminescent, radioactive, enzymatic labels or dye molecules, or other methods for detecting the formation of a complex between the antigen and the antibody or antibodies reacted therewith.

More particularly, an ELISA method can be used, wherein the wells of a microtiter plate are coated with a set of antibodies against the proteins to be tested. A biological sample containing or suspected of containing the marker protein is then added to the coated wells.

After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

The method for determining whether a microorganism is resistant to a β-lactam compound according to the invention is particularly suitable for predicting the response to a β-lactam compound in bacterial infected patient, especially in a Gram (−) bacterial infected patient.

Accordingly, an another further object of the invention relates to a method for predicting the response to a β-lactam compound of a bacterial infected patient comprising isolating the microorganism responsible for the infection and determining whether said microorganism is resistant to β-lactam compounds by performing the method as above described.

In particular embodiment when the microorganism responsible for the infection is determined as resistant the patient could be treated with another class of antibiotics.

The invention will be further illustrated by the following figures and examples. However, these examples and figures should not be interpreted in any way as limiting the scope of the present invention.

### FIGURES

FIG. 1: Antibiotic susceptibility testing to β-lactams antibiotics of E. cloacae FRI-1 expressing FRI-1.

FIG. 2: Antibiotic susceptibility testing to non β-lactam antibiotics of E. cloacae FRI-1 expressing FRI-1.

FIG. 3: Genetic environment of the bla FRI-1 gene in E. cloacae FRI-1.

### EXAMPLE

### Material and Methods

Bacterial Strains and Plasmids:

Identification of Enterobacter cloacae FRI-1 was performed by using the API 20E system (bioMérieux, Marcy l'Etoile, France). E. coli TOP10 was used as host strain for cloning and E. coli J53 (resistant to azide) as host for conjugation assays.

Antimicrobial Agents and MIC Determinations:

The antimicrobial agents and their sources have been described elsewhere. Susceptibility testing was performed by disk diffusion assay (Sanofi-Diagnostic Pasteur, Marnes-la-Coquette, France), as previously described. The minimal inhibitory concentrations (MICs) were determined by Etest (AB biodisk; Solna, Sweden) on Mueller-Hinton agar plates at 37° C. Results of susceptibility testing were recorded according to the CLSI guidelines. MBL detection tests were performed using an E-test strip (AB Biodisk).

Cloning Experiments, PCR and DNA Sequencing:

PCR screening for class A carbapenemases (bla_{KPC}, bla_{IMI} bla_{SME}), MBL encoding genes bla_{VIM}, bla_{IMP} bl, for extended-spectrum β-lactamases (ESBLs) encoding genes bla_{TEM} bla_{SHV}, and bla_{CTX-M}, and for plasmid-mediated cephalosporinase gene (bla_{CMY} bla_{ACC}), were performed as described. In order to identity the bla FRI-1 gene, blunt clonings were performed using HindIII and Sau3 A restriction enzymes using pBKCMV as a cloning vector and E. coli TOP10 as the recipient strain. In order to express in the bla_{FRI-1} gene in an identical background, cloning of this gene was performed into E. coli TOP10 as described, using the PCRBlunt® TOPO cloning kit (Invitrogen, Cergy-Pontoise, France) followed by selection on plates containing 50 μg/ml of amoxicillin and 30 μg/ml of kanamycin. The PCR amplicon encompasses the entire sequence of the bla_{FRI-1} gene. Those amplicons did not include the original promoter region of the bla_{FRI-1} gene, in order to express those genes under the control of the same promoter provided by plasmid pCR-Blunt-TOPO. Corresponding recombinant strains were used for MIC determinations.

Both strands of the cloned DNA inserts of recombinant plasmids were sequenced by using an Applied Biosystems sequencer (ABI 377). The nucleotide and deduced protein sequences were analysed with software available over the Internet from the National Center for biotechnology Information website (http://www.ncbi.nlm.nih.gov/BLAST/).

β-Lactamase Purification:

Cultures of E. coli TOP10 harboring recombinant plasmid pFRI-1 was grown overnight at 37° C. in 2 liters of TS broth containing ticarcilllin (50 μg/ml) and ticarcillin (100 μg/ml). β-Lactamase FRI-1 was purified by ion-exchange chromatography. Briefly, the bacterial suspension was pelleted, resuspended in 50 ml of 20 mM Tris-Ethanolamine buffer (pH 7.2). The pellet was then was sonicated, cleared by ultracentrifugation and treated with DNase. The extract was then dialyzed against 20 mM Bis-Tris-ethanolamine (pH 7.2) and loaded onto a preequilibrated Q-sepharose column on an AKTA purifier (GE Healthcare, USA) followed by a second Q-sepharose column using a Tris-ethaloamine buffer pH9.5. The β-lactamase-containing fractions were eluted with a linear NaCl gradient (0 to 1 M). Fractions containing the highest β-lactamase activities were pooled and subsequently dialyzed overnight against 20 mM Tris-ethanolamine buffer (pH 7.2). The β-lactamase activity was determined qualitatively using nitrocefin. The protein content was measured using the Bio-Rad DC protein assay. The purification factor was measured by comparing the activities of the FRI-1 crude extract and purified enzyme using 100 μM imipenem as substrate. The isoelectric point of the FRI-1 enzyme was determined using an electrofocalisation system on polyacrylamide gel (Clean Gel IEF, GE Healthcare, USA) and IEF standards (IEF standards, Bio-Rad).

Kinetic Studies:

Kinetic measurements (k_{cat} and Kₘ) of purified β-lactamase FRI-1 were performed spectrophotometrically as described previously and compared to those previously published for class A carbapenemases.

Plasmid Content, Conjugation Assays, and Transformation:

Plasmid DNAs of E. cloacae FRI-1 was extracted by using the Kieser method. E. coli NCTC50192, harboring four plasmids of 154, 66, 48 and 7 kb, was used as the size marker for plasmids. Plasmid DNAs were analyzed by agarose gel electrophoresis as described previously. Direct transfer of the β-lactam resistance markers into azide-resistant E. coli J53 was attempted by liquid mating out assays at 37° C. Selection of the transformants was performed on agar plates supplemented with aztreonam (50 μg/ml), azide (100 μg/ml) and nalidixic acid (20 μg/ml). Plasmid encoding the FRI-1 determinant was typed according to known typing scheme.

Results:

Characteristics of E. cloacae FRI-1:

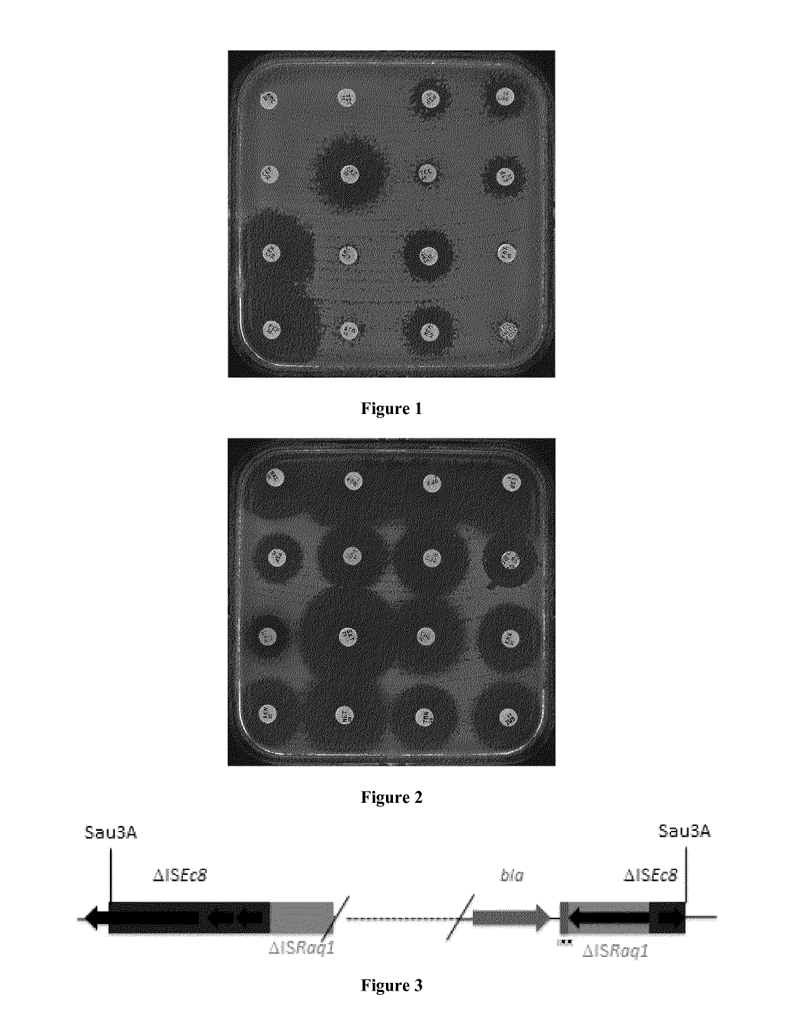

Isolate E. cloacae FRI-1 was resistant or had a decreased susceptibility to all β-lactams, including imipenem, meropenem, and ertapenem (FIG. 1). This strain was mostly susceptible to non-β-lactam antibiotics (FIG. 2).

Genetic Support of the bla_{FRI-1} Gene:

Plasmid analysis identified two plasmids of ca. 150-160 kb and ca. 60-70 kb, in E. cloacae FRI-1. Conjugation experiments failed to produce E. coli transconjugants exhibiting using E. coli INE-1 s as the donor. This indicates that the FRI-1 plasmid was neither self-conjugative nor mobilizable by one of the natural plasmids contained in E. cloacae FRI-1. A single FRI-1 plasmid was obtained after electrotransformation experiments of ca. 160-170 kb in size. This plasmid was not typeable according to typing plasmid scheme and did not confer any additional resistance markers. Using a sequencing primer walking strategy, the analysis of the genetic environment of the bla_{FRI-1} gene in isolate FRI-1 showed that it was bracketed by an ISRaq1 element (FIG. 3).

Characterization of FRI-1:

β-Lactamase FRI-1 is an Ambler class A β-lactamase. However it differs significatively from known carbapenemases. This 293 amino acid protein shared 55, 54, 54, 53, 51% amino-acid identity with NMC-A, IMI-1, Sme-1/Sme-1, SFC-1 and KPC-2. It was therefore distantly related to non-Ambler class A carbapenemases such as NDM-1, IMP-1, VIM-1 and OXA-48. Expression of the bla_{FRI-1} gene in E. coli TOP10 conferred as expected reduced susceptibility to all β-lactams tested (Table A). The highest MIC values were those observed for ceftazidime and aztreonam. As observed for class A carbapenemases, addition of a fixed concentration of clavulanic acid (4 ug/ml) decreased the MIC values for all antibiotic tested. Similar results were obtained after addition of tazobactam (4 μg/ml) which is also a classical β-lactamase inhibitor of class A carbapenemase (data not shown).

FRI-1 was purified to near homogeneity (>90%) as determined by SDS-PAGE analysis, and the purification factor was estimated to be 40-fold. Its molecular weight was ca. 30 kDa and its pI value was 8.66. β-Lactamase FRI-1 hydrolyzed all tested β-lactams including significantly the carbapenems tested (Table B).

TABLE A
MIC values of several β-lactams.

| | | E. coli TOP10 | |
|---|---|---|---|
| | E. cloacae | cloned FRI-1 | |
| | FRI-1 | gene | E. coli TOP10 |
| Imipenem | 8 | 0.75 | 0.2 |
| Imipenem + Clav | 4 | 0.5 | 0.2 |
| Ertapenem | 24 | 0.12 | 0.006 |
| Ertapenem + Clav | 8 | 0.03 | 0.006 |
| Meropenem | 3 | 0.12 | 0.02 |
| Meropenem + clav | 2 | 0.03 | 0.02 |
| Ceftazidime | 6 | 1 | 0.2 |
| Ceftazidime + clav | 3 | 0.38 | 0.01 |
| Cefotaxime | 1 | 0.25 | 0.05 |
| Aztreonam | 256 | 256 | 0.1 |
| Aztreonam + clav | 128 | 8 | 0.1 |
| Cefepime | 0.38 | 0.06 | 0.02 |
| Cefpirome | 1.5 | 0.1 | 0.05 |

TABLE B
Kinetic parameters of purified FRI-1

| | FRI-1 | | |
|---|---|---|---|
| | Kₘ | K_{cat} | K_{cat}/Km |
| β-Lactam | (μM) | (sec⁻¹) | (μM⁻¹/sec⁻¹) |
| Penicillin G | 567 | 1,060 | 0.21 |
| Amoxicillin | >5,000 | — | — |
| Ticarcillin | 393 | 120 | 305 |
| Piperacillin | >3,000 | — | — |
| Cefalotin | >3,000 | — | — |
| Cefotaxime | >5,000 | — | — |
| Cefepime | 3,400 | 28 | 8 |
| Moxalactam | >2,600 | — | — |
| Aztreonam | >5,000 | — | — |
| Imipenem | 1,614 | 1,800 | 1,100 |
| Ertapenem | 98 | 150 | 1,500 |
| Meropenem | 70 | 45 | 650 |

### REFERENCES

Throughout this application, various references describe the state of the art to which this invention pertains. The disclosures of these references are hereby incorporated by reference into the present disclosure.
Ambler R P. 1980, Philos Trans R Soc Lond B Biol Sci 289:321-31
Aubron C, Poirel L, Ash R J, Nordmann P. 2005. Emerg Infect Dis 11:260-4
Carattolli A, Bertini A, Villa L, Falbo V, Hopkins K L, Threlfall E J. 2005. J Microb Methods 63:219-228.
Chang A C, Nunberg J H, Kaufman R J, Erlich H A, Schimke R T, Cohen S N. 1978 Nature 275(5681):617-24.
Chevray, Nathans, 1991. Proc. Natl. Acad. Sci. USA 89: 5789-5793
Chien et al. 1991. Proc. Natl. Acad. Sci. USA 88: 9578-9582
De Boer L W, Rude R E, Kloner R A, Ingwall J S, Maroko P R, Davis M A, Braunwald E. 1983. Proc Natl Acad Sci USA. 80:5784-8.
Fields, Song. 1989. Nature (London), 340: 245-246.
Goeddel D V, Heyneker H L, Hozumi T, Arentzen R, Itakura K, Yansura D G, Ross M J, Miozzari G, Crea R, Seeburg P H. 1979. Nature.; 281:544-8.
Goeddel D V, Shepard H M, Yelverton E, Leung D, Crea R, Sloma A, Pestka S. 1980. Nucleic Acids Res. 8:4057-74.
Merrifield, J. Am. Chem. Soc., 1963. 85: 2149-2154
Munoz-Price L S et al. 2013. Lancet Infect Dis. In press
Naas T, Nordmann P. 1994. Proc. Natl Acad Sci USA 91:7693-7697.
Poirel L., Walsh T R., Cuvillier V, Nordmann P. 2011. Diagn. Microbiol. Infect. Dis. 70:119-123.
Nordmann P. 2013. Med Mal Infect, in press
Nordmann P, Cuzon G, Naas T. 2009. Lancet Infect Dis 9:228-36
Nordmann P, Dortet L, Poirel L. 2012 Trends Mol Med 18:263-272
Queenan A M, Bush K. 2007. Clin Microb Rev 20:440-458.
Sambrook J, Fritsch E F, Maniatis T. 1989. Molecular cloning: a laboratory manual, 2^{nd} ed. Cold Spring Harbor Laboratory Press, Cold Spring Harbor, N.Y. Stewart et al., Solid-Phase Peptide Synthesis (W.H. Freeman Co.: San Francisco, Calif., 1969);

1. A FRI-1 carbapenemase comprising an amino acid sequence defined by SEQ ID NO: 1 or having at least 80% amino acid sequence identity with the amino acid sequence defined by SEQ ID NO: 1.

2. A nucleic acid sequence encoding a carbapenemase comprising an amino acid sequence defined by SEQ ID NO: 1 or having at least 80% amino acid sequence identity with the amino acid sequence defined by SEQ ID NO: 1.

3. The nucleic acid sequence according to claim 2 which is defined by SEQ ID NO: 2.

4. A method for detecting the presence of a FRI-1 carbapenemase comprising an amino acid sequence defined by SEQ ID NO: 1 or having at least 80% amino acid sequence identity with the amino acid sequence defined by SEQ ID NO: 1 in a biological sample, comprising a step of contacting said biological sample with a binding reagent specific for said FRI-1 carbapenemase.

5. An antibody or an aptamer which specifically binds to a FRI-1 carbapenemase comprising an amino acid sequence defined by SEQ ID NO: 1 or having at least 80% amino acid sequence identity with the amino acid sequence defined by SEQ ID NO:1.

6. A kit comprising
an antibody or an aptamer which specifically binds to a FRI-1 carbapenemase comprising an amino acid sequence defined by SEQ ID NO: 1 or having at least 80% amino acid sequence identity with the amino acid sequence defined by SEQ ID NO:1; or
a probe or a set of primers which specifically hybridizes to a nucleic acid sequence encoding the FRI-1 carbapenemase.

7. A method for detecting the presence of a nucleic acid sequence encoding a carbapenemase comprising the amino acid sequence defined by SEQ ID NO: 1 or having at least 80% amino acid sequence identity with the amino acid sequence defined by SEQ ID NO:1 in a biological sample, comprising a step of contacting said biological sample with a nucleic acid molecule which specifically hybridizes to said nucleic acid sequence.

8-9. (canceled)

10. A probe or a set of primers which specifically hybridizes to a nucleic acid sequence encoding a FRI-1 carbapenemase comprising an amino acid sequence defined by SEQ ID NO: 1 or having at least 80% amino acid sequence identity with the amino acid sequence defined by SEQ ID NO:1.

11. The method according to claim 10, said method comprising the steps of:
(i) providing a FRI-1 carbapenemase comprising an amino acid sequence defined by SEQ ID NO: 1 or having at least 80% amino acid sequence identity with the amino acid sequence defined by SEQ ID NO:1 and a substrate of the FRI-1 carbapenemase
(ii) bringing the candidate substance to be tested into contact with the FRI-1 carbapenemase and the substrate of step i);
(iii) determining the activity of said FRI-1 carbapenemase in presence of the candidate substance to be tested;
(iv) comparing the catalytic activity of said carbapenemase determined at step iii) with the catalytic activity of said carbapenemase in the absence of said candidate substance; and
(v) positively selecting the candidate substance that inhibits the catalytic activity of said carbapenemase.

12. A method for determining whether a microorganism is resistant to a β-lactam compound comprising the step of
detecting in said microorganism the presence of a nucleic acid encoding a FRI-1 carbapenemase comprising an amino acid sequence defined by SEQ ID NO: 1 or having at least 80% amino acid sequence identity with the amino acid sequence defined by SEQ ID NO:1, wherein the presence of said nucleic acid is indicative that said microorganism is resistant to β-lactams.

13. A method for predicting whether or not a patient that is infected with a microorganism will respond to treatment with a β-lactam compound, comprising
isolating the microorganism responsible for the infection and
determining whether said microorganism is resistant to β-lactam compounds by detecting in said microorganism the presence of a nucleic acid encoding a FRI-1 carbapenemase comprising an amino acid sequence defined by SEQ ID NO: 1 or having at least 80% amino acid sequence identity with the amino acid sequence defined by SEQ ID NO:1, wherein the presence of said nucleic acid is indicative that said microorganism is resistant to β-lactams, and that said patient will not respond to treatment with a β-lactam compound.


SEQUENCE LISTING (2 sequences)

SEQ ID NO: 1 - protein, 293 aa, Enterobacter cloacae
MFFFKKGASTFIFLLCLPLNSFASQVINSVEEMRELETSFGGRIGVYILNPKNGKEFAYRQDERFPLCSSFKAFLAASVLKRTQEKSVSLDDMVEYSGRVMEKHSPVSEKYRKTGASVQTLAKAAIQYSDNGASNLLMERYIGGPEGLTAFMRSTGDTDFRLDRWELELNTAIPGDERDTSTPKAVAMSLKNIAFGSVLDAKNKSLLQEWLKGNTTGNARIRAAVPDKWVVGDKTGTCGFYGTANDVAILWTDANSPAVMAVYTTRPNQNDKHDEAVIKNAAKIAIKAVYGSY

SEQ ID NO: 2 - DNA, 881 bp, Enterobacter cloacae
atgtttttttttaaaaaaggtgcaagtacatttatttttttgctctgtcttccattgaactcattcgcctctcaggtaattaatagtgttgaggaaatgagggaattggaaacttcttttggggggcggataggtgtttatattttaaacccaaaaaatgggaaagaatttgcctacagacaagatgagagatttcctttatgtagttcatttaaggcgttcctcgctgcatccgtattaaaaagaactcaggagaaatctgtttctcttgatgatatggtggaatattctggacgtgttatggaaaagcattctcctgtgtcagaaaaataccgtaaaacaggagcaagcgtgcagactttggccaaggcagcaattcagtatagtgacaatggagcttctaatctattaatggaaagatacataggaggtcctgagggtttgactgcatttatgcggtcaacgggagacactgacttcaggcttgatcgttgggaattagaattaaacacagctattccaggcgatgaacgagatacttcaactccaaaagcagtggcaatgagccttaaaaatattgcttttggttcagtactcgatgctaaaaataaatcattgctgcaggaatggcttaaaggcaacactactggtaatgcgcgaattagagctgcggttccagataagtgggttgttggcgataaaacaggcacctgtggtttttatggtacagccaatgatgttgctattttatggacagacgccaattcacctgcagttatggctgtctacacaacacgtcctaatcaaaacgacaaacatgacgaagcagtaattaaaaatgctgcaaaaatagctataaaggcagtttatggaagttataa